(19) United States
(12) Patent Application Publication
(10) Pub. No.: US 2026/0165927 A1
(43) Pub. Date: Jun. 18, 2026

(54) HAIR CARE COMPOSITION COMPRISING FRIZZ REDUCTION AGENT SUCH AS SALICYLIC ACID

(71) Applicant: The Procter & Gamble Company, Cincinnati, OH (US)

(72) Inventors: Gemma Zoe Braganza, Singapore (SG); Jennifer Mary Marsh, Mason, OH (US); Zhicai Zhou, Singapore (SG)

(21) Appl. No.: 19/416,004

(22) Filed: Dec. 11, 2025

Related U.S. Application Data
(60) Provisional application No. 63/733,495, filed on Dec. 13, 2024.

(51) Int. Cl. A61K 8/365; A61K 8/34; A61K 8/42; A61K 8/894; A61Q 5/12

(52) U.S. Cl. CPC A61K 8/365; A61K 8/342; A61K 8/345; A61K 8/42; A61K 8/894; A61Q 5/12; A61K 2800/262; A61K 2800/31; A61K 2800/805

(57) ABSTRACT

The present disclosure relates to a hair care composition comprising: (a) from about 1.2% to about 5% of a cationic surfactant; (b) from about 3% to about 20% of a fatty alcohol selected from the group of consisting of myristyl alcohol, cetyl alcohol, and mixtures thereof, (c) from about 50% to about 90% of a solvent system selected from the group consisting of di-propylene glycol, propylene glycol, pentylene glycol, hexylene glycol, 1,2-hexanediol, sugar alcohol, PEG-4, and mixtures thereof; wherein the composition contains less than about 5% of a C1-C3 mono-alcohol; wherein the composition further comprises (d) from about 0.5% to about 20% of a frizz reduction agent which is an acidic material such as citric acid, succinic acid and/or salicylic acid. The present disclosure provides a hair care composition: which provide, in addition to frizz reduction, good conditioning benefit especially during application such as the application to wet hair such as wet detangling, while not weighing down the hair and/or making the hair heavy and/or greasy.

### FIELD OF THE INVENTION

The present disclosure relates to a hair care composition comprising: The present disclosure relates to a hair care composition comprising: (a) from about 1.2% to about 5% of a cationic surfactant; (b) from about 3% to about 20% of a fatty alcohol selected from the group of consisting of myristyl alcohol, cetyl alcohol, and mixtures thereof, (c) from about 50% to about 90% of a solvent system selected from the group consisting of di-propylene glycol, propylene glycol, pentylene glycol, hexylene glycol, 1,2-hexanediol, sugar alcohol, PEG-4, and mixtures thereof, wherein the composition contains less than about 5% of a C₁-C₃ mono-alcohol; wherein the composition further comprises (d) from about 0.5% to about 20% of a frizz reduction agent which is an acidic material such as citric acid, succinic acid and/or salicylic acid. The present disclosure provides a hair care composition: which provide, in addition to frizz reduction, good conditioning benefit especially during application such as the application to wet hair such as wet detangling, while not weighing down the hair and/or making the hair heavy and/or greasy.

### BACKGROUND OF THE INVENTION

Hair frizz is described by consumers as the appearance of unruly fibers at the top of the scalp and tips of hair as well as an increased volume through the bulk of the hair. Generally, they see this frizz on days the level of moisture in the air is high. The appearance of frizz is undesired and it is often associated with a loss of shine and smoothness, which are associated with a perception of poor hair health. The common accepted mechanism causing frizz in high humid environments is that at high humidity water penetrates into hair and changes the chemical bonding interactions inside the hair. During styling, the consumer will create a ‘wet set’ where hair is blow dried or flat ironed to create the desired shape. During drying, water is evaporated from hair and hydrogen bonds (and other bonding interactions) are formed between the protein chains holding the style in place. As moisture diffuses into hair the hydrogen bonds (and other bonding interactions) are broken and hair returns back to its natural shape. For consumers who straighten their hair by blow drying or flat ironing, this return to a curled style is associated with a loss of alignment and increased volume. In addition, at high moisture levels in hair the fiber diameter increases which also increases the overall volume of hair.

The typical strategy to prevent frizz is to formulate leave-on or rinse-off products with surface-depositing materials such as silicone, oils, conditioning silicone etc. which make hair more hydrophobic and decrease inter-fiber interactions. At high levels these materials can also provide increased cohesive forces holding fibers together to prevent frizz from occurring. With these materials depositing on the hair surface a greasy look and feel is typically experienced, which is an undesired trade-off of frizz reduction.

Consequently, a need exists for hair products that are effective in controlling frizz and at the same time delight consumers by providing hair that do not have sticky or greasy feel.

For example, US2018/0289603 A (P&G) discloses a non-aqueous hair leave-on composition for hair frizz reduction comprising from about 0.15% to about 12.0% of a moisture control material or mixture of moisture control materials. This US publication discloses in Tables 7 and 8, compositions containing 1% of salicylic acid, at least about 70% of at least one selected from dimethicone, cyclopentasiloxane, and isododecane. However, such non-aqueous compositions containing higher amount of oils still weigh down the hair and/or make the hair heavy and/or greasy.

US2016/158135 A (P&G) discloses a rinse-off composition for hair frizz reduction comprising from about 0.2% to about 20.0% of a moisture control material such as salicylic acid or mixture of moisture control materials. This US publication discloses in Table 1, compositions containing 1% of salicylic acid, about 3% of a mixture of BTMAC (cationic surfactant)/IPA, about 2% of cetyl alcohol, about 4.6% of stearyl alcohol, and at least 80% of water. However, the present inventors have surprisingly found that, when salicylic acid is included in such aqueous compositions, salicylic acid tends to negatively affect the formation and/or stability of thick cream or gel such as gel network. Thick cream or gel such as gel network is expected to be formed by cationic surfactant and fatty alcohol in aqueous compositions and is thought to provide good wet conditioning such as wet detangling.

WO2023/172867 (P&G) discloses an anhydrous hair care composition comprising from about 5% to about 50% of a cationic surfactant; a fatty alcohol; and at least about 40% of a solvent system comprising a mono-alcohol such as ethanol and isopropyl alcohol. This WO publication discloses that the consumer may dilute this concentrated hair care composition with water in a ratio from about 1:1 to about 1:20 to form a thick cream or gel, and this hydrated composition is then used in any known manner as a hair conditioner. This WO publication also discloses a composition as a comparative example C4 comprising 7.75% SAPDMA (Stearamidopropyl Dimethylamine), 10.25% of Cetyl alcohol, 4.5% of stearyl alcohol and 2.5% of salicylic acid 75% of Dipropylene glycol, wherein no mono-alcohol such as ethanol and isopropyl alcohol is used in the composition. However, the WO publication describes that this composition is not a single phase liquid, and there is a solid precipitate. It may be desired by consumers to have hair care products with reduced amount of C1-C3 mono-alcohol such as ethanol in view of reduced irritation and/or reduced dry perception, while still being a single phase liquid without solid precipitation.

Thus, there remains a need for hair care composition: which provide, in addition to frizz reduction, good conditioning benefit especially during application such as the application to wet hair such as wet detangling, while not weighing down the hair and/or making the hair heavy and/or greasy; which contains reduced amount of C1-C3 mono-alcohol in view of reduced irritation and/or reduced dry perception; which is a single phase liquid without solid precipitate, and/or has a clear or transparent appearance.

### SUMMARY OF THE INVENTION

The present disclosure is directed to a hair care composition comprising:
(a) from about 1.2% to about 5% of a cationic surfactant;
(b) from about 3% to about 20% of a fatty alcohol selected from the group of consisting of myristyl alcohol, cetyl alcohol, and mixtures thereof;
(c) from about 50% to about 90% of a solvent system selected from the group consisting of di-propylene glycol, propylene glycol, pentylene glycol, hexylene glycol, 1,2-hexanediol, sugar alcohol, PEG-4, and mixtures thereof;
wherein the composition contains less than about 5% of a C1-C3 mono-alcohol;
wherein the composition further comprises (d) from about 0.5% to about 20% of a frizz reduction agent.

Preferably the frizz reduction agent is citric acid, succinic acid and/or at least one of those having the structure:

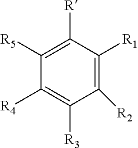

wherein R′ is —COOY, sulfonic acid, or —C═CH—COOY, Y is hydrogen or a metal ion, R₁, R₂, R₃, R₄, R₅ is hydrogen, methyl, ethyl, propyl, vinyl, allyl, methoxy, ethoxy, hydroxyl, halogen, sulfate, sulfonate, nitro, or —CH═CH—COOR, and wherein the frizz reduction agent is an acidic material and further wherein the frizz reduction agent has a % Protein binding higher than 20 and Molecular Volume lower than 500 and Partition coefficient octanol to water (log P) lower than 3 and hydrogen binding higher than 10 and pKa lower than 5.0.

The present disclosure provides a hair care composition: which provide, in addition to frizz reduction, good conditioning benefit especially during application such as the application to wet hair such as wet detangling, while not weighing down the hair and/or making the hair heavy and/or greasy; which contains reduced amount of C1-C3 mono-alcohol in view of reduced irritation and/or reduced dry perception; which is a single phase liquid without solid precipitate, and/or has a clear or transparent appearance; and which can be used in a convenient manner, for example, without such dilution with water before use.

By the use of higher % of such specific non-aqueous solvent, the composition reduces negative affection of salicylic acid to the formation and/or stability of thick cream or gel such as gel network. Thick cream or gel such as gel network is expected to be formed by cationic surfactant and fatty alcohol with the presence of water and is thought to provide good wet conditioning such as wet detangling. Without being limited to the theory, it is believed that, by applying this composition to wet hair without diluting with water before use, the composition provides frizz reduction and also provides wet conditioning such as wet detangling, by formation of thick cream or gel such as gel network on the hair.

By the use of higher % of such specific non-aqueous solvent, the composition can dissolve the cationic surfactant and fatty alcohols without the use of oil solvents such as dimethicone, cyclopentasiloxane, and isododecane. Thus, the composition provides the above conditioning benefits while not weighing down the hair and/or making the hair heavy and/or greasy.

Also, by the use of the specific fatty alcohol together with the use of higher % of such specific non-aqueous solvent, the composition can be a single phase liquid without solid precipitate, and/or have a clear or transparent appearance, without the use of C1-C3 mono-alcohol, thus the composition can provide reduced irritation and/or reduced dry perception.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

Q.S. herein means up to 100%.

### Composition

The hair care composition comprises:
(a) from about 1.2% to about 5% of a cationic surfactant;
(b) from about 3% to about 20% of a fatty alcohol selected from the group of consisting of myristyl alcohol, cetyl alcohol, and mixtures thereof,
(c) from about 50% to about 90% of a solvent system selected from the group consisting of di-propylene glycol, propylene glycol, pentylene glycol, hexylene glycol, 1,2-hexanediol, sugar alcohol, PEG-4, and mixtures thereof,
wherein the composition contains less than about 5% of a C1-C3 mono-alcohol;
wherein the composition further comprises (d) from about 0.5% to about 20% of a frizz reduction agent.

Preferably the frizz reduction agent is citric acid, succinic acid and/or at least one of those having the structure:

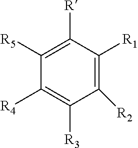

wherein R′ is —COOY, sulfonic acid, or —C═CH—COOY, Y is hydrogen or a metal ion, R₁, R₂, R₃, R₄, R₅ is hydrogen, methyl, ethyl, propyl, vinyl, allyl, methoxy, ethoxy, hydroxyl, halogen, sulfate, sulfonate, nitro, or —CH═CH—COOR, and wherein the frizz reduction agent is an acidic material and further wherein the frizz reduction agent has a % Protein binding higher than 20 and Molecular Volume lower than 500 and Partition coefficient octanol to water (log P) lower than 3 and hydrogen binding higher than 10 and pKa lower than 5.0.

Preferably, the composition contains less than 1%, more preferably less than 0.5%, less than 0.1%, still more preferably 0% of fatty alcohols having 18 or more carbons.

Preferably, in the composition, the fatty alcohol contains from about 25% to about 100%, more preferably from about 30% to about 80%, from about 35% to about 60%, from about 40% to about 50% by weight of the fatty alcohol, of myristyl alcohol.

### Product Forms and Method of Use

The composition may be anhydrous, in view of having a clear appearance and low viscosity. Anhydrous herein means that the composition comprises at most 2% total water and no added water, meaning at most 2% bound water.

The hair care composition may be a low viscosity liquid. The composition preferably has a viscosity of from about 1 to about 300 cPs, more preferably from about 1 to about 100 cPs, in view of having a light texture. The liquid may be a single homogenous phase.

The consumers may dispense the composition from a container to their dry or wet palms and apply to their dry or wet hair, preferably to their wet hair. There may be no intentional dilution of the composition with water after dispensing and before applying to the hair. Such intentional dilution means, for example, mixing the composition with water in a ratio more than 1:0.8, preferably more than 1:0.5.

During the application, the composition is mixed with water, and may form a thick cream or gel. The hair care composition may instantly form a conditioner gel network when mixed with water. The composition preferably has a shear stress, when mixed 1:1 with water, at a shear rate of 950s⁻¹, of from about 50 Pa to about 400 Pa in view of providing a good conditioning benefits especially wet detangling. Then, the composition may be rinsed or not be rinsed.

The composition is preferably clear or transparent, in view of providing the consumers with a perception of “not weighing down the hair and/or making the hair heavy and/or greasy”. To be transparent or clear is defined as a percent transmittance (% T) of at least 80% transmittance at 600 nm, according to the Clarity Assessment Test Method herein. Upon the addition of water, the composition may no longer be transparent.

A method of using the composition, comprising the following steps:
Dispense the composition from a container;
Apply the composition to wet hair;
preferably wherein, before applying to the wet hair, the method does not contain a step to mix the composition with water in a ratio more than 1:0.8, preferably more than 1:0.5. After applying, the composition may be rinsed or may not be rinsed.

### Frizz Reduction Agent

The composition comprises a frizz reduction agent. The frizz reduction agent is included in the composition, at a level by weight of the composition, of from about 0.2% to about 20%, in an embodiment from about 0.5% to about 8.0%, and in a further embodiment from about 0.5% to about 5.0%. and in yet a further embodiment from about 1.0% to about 3.0%.

The frizz reduction agent can be citric acid and/or succinic acid. Alternatively or concurrently, the frizz reduction agents can be at least one of the ingredients which are also called as moisture control materials, and are those having the structure:

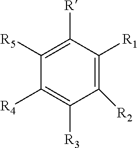

wherein R′ is —COOY, sulfonic acid, or —C═CH—COOY, Y is hydrogen or a metal ion, R₁, R₂, R₃, R₄, R₅ is hydrogen, methyl, ethyl, propyl, vinyl, allyl, methoxy, ethoxy, hydroxyl, halogen, sulfate, sulfonate, nitro, or —CH═CH—COOR, and wherein the moisture control material is an acidic material and further wherein the moisture control material has a % Protein binding higher than 20 and Molecular Volume lower than 500 and Partition coefficient octanol to water (log P) lower than 3 and hydrogen binding higher than 10 and pKa lower than 5.0.

Examples of such materials are shown below: Polar, acidic compounds with the following properties: Protein Binding (PB)>20, preferably 40 or more, more preferably 43 or more, AND Molecular Volume (Mol. Vol). <500 AND log P <3 AND Hydrogen-binding (H-binding) >10 AND pKa <5.0, wherein PB is % protein binding, Mol. Vol is molecular volume (in Å3); log P is n-octanol/water partition coefficients. These properties can be calculated using Volsurf software (http://www.moldiscovery.com/soft_volsurfphp). H-bond is the energy from hydrogen bonds between molecules from Hansen Solubility Parameters and pKa value is a logarithmic measure of the acid dissociation constant.

| | | Mol. | log | | H-bond | % Water |
|---|---|---|---|---|---|---|
| Name (1% wt/vol) | PB | Vol. | P | pKa | (MPa\hat{ }1/2) | Reduction |
| 2,4-Dihydroxybenzoic acid | 28 | 324 | 1.5 | 3.2 | 23 | 30 |
| 3-Hydroxybenzoic Acid | 38 | 314 | 1.6 | 4.3 | 20 | 20 |
| Gallic acid | 23 | 337 | 0.9 | 4.4 | 23 | 15 |
| 3-Aminobenzoic acid | 41 | 326 | 0.9 | 3.6 | 16 | 12 |
| 4-Aminobenzoic acid | 42 | 323 | 0.9 | 3.5 | 16 | 12 |
| 2,5-Dihydroxybenzoic acid | 31 | 329 | 1.6 | 2.9 | 23 | 27 |
| 3,4-Dihydroxybenzoic acid | 27 | 327 | 0.9 | 4.4 | 23 | 20 |
| 3,5-Dihydroxybenzoic acid | 27 | 327 | 0.9 | 4.1 | 23 | 15 |
| 2,6-Dihydroxybenzoic acid | 37 | 326 | 1.6 | 2.1 | 23 | 35 |
| 5-Chlorosalicylic acid | 56 | 361 | 2.3 | 3.0 | 21 | 28 |
| Salicylic acid | 44 | 320 | 2.1 | 3.1 | 20 | 18 |
| Trans-Ferulic Acid | 50 | 451 | 1.5 | 4.5 | 19 | 6 |
| p-Coumaric acid | 46 | 391 | 1.6 | 4.5 | 20 | 8.8 |
| 4-Hydroxybenzenesulphonic | 55 | 271 | 1.5 | 2.7 | 22 | 26 |
| acid | | | | | | |
| 3-Chloro-4-hydroxybenzoic | 49 | 356 | 2.1 | 4.1 | 20 | 11 |
| acid | | | | | | |
| 3,5-Dichloro-4-hydroxybenzoic | 51 | 397 | 2.8 | 3.8 | 20 | 15 |
| acid | | | | | | |
| 2,5 Dihydroxyterephthalic acid | 20 | 375 | 1.1 | 2.1 | 22 | 18 |
| 3-Aminophenol | 45 | 284 | 0.6 | 4 | 17 | 14 |
| 3-Hydroxyanilinium chloride | 32 | 280 | 0.6 | 4 | 17 | 16 |
| 2-Aminophenol | 49 | 288 | 1.0 | 4 | 17 | 14 |
| 4-Aminophenol | 39 | 284 | 0.6 | 4 | 17 | 10 |
| N-4-Hydroxyphenylglycine | 37 | 388 | 1.3 | 3 | 13 | 15 |

Among them, preferred are salicylic acid, 5-chlorosalicylic acid, p-Coumeric acid, and ferulic acid, and more preferred is salicylic acid, in view of: frizz reduction benefit; compatibility with other ingredients such as solvent, cationic surfactant and fatty alcohols; commercial availability; and maybe additional skin/scalp benefits.

### Solvent System

The composition comprises from about 50% to about 90%, preferably from about 80% to about 90% of a solvent system, in view of the balance between conditioning benefit and product appearance such as being a single phase liquid without solid precipitate and/or having a clear or transparent appearance.

The solvent system is selected from the group consisting of di-propylene glycol, propylene glycol, pentylene glycol, hexylene glycol, 1,2-hexanediol, sugar alcohol, PEG-4, and mixtures thereof, preferably selected from the group consisting of di-propylene glycol, propylene glycol, pentylene glycol, hexylene glycol, 1,2-hexanediol, and mixtures thereof, in view of maintaining the product appearance under a variety of conditions whilst providing light texture and lower viscosity. More preferably the solvent system contains from about 50% to about 100% from about 75% to about 100%, by weight of the solvent system, of dipropylene glycol, in view of maintaining the product appearance whilst providing light texture and lower viscosity.

### C1-C3 Mono-Alcohol

The composition contains less than about 5%, preferably less than 3%, more preferably less than 1% of a C1-C3 mono-alcohol, in view of providing reduced irritation and/or reduced dry perception. C1-C3 mono-alcohols are those having 1-3 carbons, and are those selected from ethanol and isopropanol.

### Cationic Surfactant System

The hair care composition described herein comprise a cationic surfactant system. The cationic surfactant system can be one cationic surfactant or a mixture of two or more cationic surfactants.

The cationic surfactant system can be selected from: mono-long alkyl quaternized ammonium; a combination of mono-long alkyl quaternized ammonium and di-long alkyl quaternized ammonium; mono-long alkyl amidoamine; a combination of mono-long alkyl amidoamine and di-long alkyl quaternized ammonium; a combination of mono-long alkyl amindoamine and mono-long alkyl quaternized ammonium. Among them, preferred is mono-long alkyl amidoamine, in view of solubility in the solvent system while providing the conditioning benefits.

The cationic surfactant system can be included in the hair care composition at a level of from about 1.2% to about 5%, preferably from about 1.5% to about 5%, more preferably from about 2% to about 5%, by weight of the hair care composition, in view of providing conditioning benefits whilst reducing any potential irritation. For leave-on use, it is preferred that the composition contains up to about 2% of mono-long alkyl amidoamine, while the composition still can contain up to about 5% of cationic surfactants in total.

The cationic surfactants are preferably used in combination with acid, thus, in a salt form.

### Mono-Long Alkyl Amidoamines, and Salts Thereof

Mono-long alkyl amidoamines are also suitable as cationic surfactants. Primary, secondary, and tertiary fatty amidoamines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al.

Preferably, in the composition, the cationic surfactant is a mono-long alkyl amidoamine such as stearamidopropyldimethylamine (SAPDMA), and the composition further comprises an acid which is soluble in the solvent system, in view of providing improved conditioning benefits while having the product appearance. The acid is selected from the group consisting of: salicylic acid, azelaic acid, malic acid, maleic acid, mandelic acid, succinic acid, fumaric acid, glyoxylic acid, glycolic acid, lactic acid, citric acid, cinnamic acid, 1-glutamic acid, preferably the acid is selected from the group consisting of: salicylic acid, azelaic acid, citric acid, maleic acid, mandelic acid, succinic acid, malic acid, and mixtures thereof. More preferably, the acid is selected from the group consisting of: salicylic acid, azelaic acid and mixtures thereof, in view of providing conditioning benefits especially frizz reduction. The weight ratio between the mono-long alkyl amidoamine and the acid is preferably from about 1:0.1 to about 1:5, more preferably from about 1:0.3 to about 1:2, still more preferably from about 1:0.3 to about 1:1, in view of providing conditioning benefits especially frizz reduction.

### Mono-Long Alkyl Quaternized Ammonium Salt

The monoalkyl quaternized ammonium salt cationic surfactants useful herein are those having one long alkyl chain which has from 12 to 30 carbon atoms, from 16 to 24 carbon atoms, and in one embodiment at C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

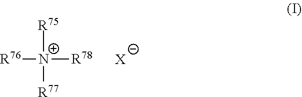

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ can be selected from an alkyl group of from 12 to 30 carbon atoms, from 16 to 24 carbon atoms, from 18 to 22 carbon atoms, an/or 22 carbon atoms; the remainder of R⁷⁵, R⁷⁶, R⁷⁷ and R⁷⁸ are independently selected from CH₃, C2H₅, C2H₄OH, and mixtures thereof, and X is selected from the group consisting of Cl, Br, CH₃OSO₃, C2H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Di-Long Alkyl Quaternized Ammonium Salt

Di-long alkyl quaternized ammonium salt can be combined with a mono-long alkyl quaternized ammonium salt or mono-long alkyl amidoamine salt. It is believed that such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts are used at a level such that the wt % of the dialkyl quaternized ammonium salt in the cationic surfactant system is in the range of from about 10% to about 50%, and/or from about 30% to about 45%.

The dialkyl quaternized ammonium salt cationic surfactants useful herein are those having two long alkyl chains having 12-30 carbon atoms, and/or 16-24 carbon atoms, and/or 18-22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

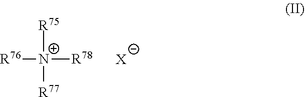

wherein two of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ can be selected from an alkyl group of from 12 to 30 carbon atoms, from 16 to 24 carbon atoms, from 18 to 22 carbon atoms, and/or 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂HSOSO₃, and mixtures thereof.

Such dialkyl quaternized ammonium salt cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride. Such dialkyl quaternized ammonium salt cationic surfactants also include, for example, asymmetric dialkyl quaternized ammonium salt cationic surfactants.

### Fatty Alcohol

The composition comprises from about 3% to about 20%, preferably from about 3.5% to about 15%, more preferably from about 5% to about 15% of a fatty alcohol, in view of the balance between conditioning benefit and product appearance such as being a single phase liquid without solid precipitate and/or having a clear or transparent appearance.

The fatty alcohols are selected from the group of consisting of myristyl alcohol, cetyl alcohol, and mixtures thereof, in view of maintaining the stability of the product appearance in a broader range of temperatures, whilst providing conditioning benefits.

Preferably, in the composition, the fatty alcohol contains from about 25% to about 100%, more preferably from about 30% to about 80%, from about 35% to about 60%, from about 40% to about 50% by weight of the fatty alcohol, of myristyl alcohol. in view of maintaining the stability of the product appearance in a broader range of temperatures whilst providing conditioning benefits.

Preferably, the composition contains less than 1%, more preferably less than 0.5%, less than 0.1%, still more preferably 0% of fatty alcohols having 18 or more carbons. in view of the product appearance such as being a single phase liquid without solid precipitate and/or having a clear or transparent appearance.

### Total Amount of the Cationic Surfactant and the Fatty Alcohol

The total amount of the cationic surfactant and the fatty alcohol is preferably from about 4.2% to about 22%, more preferably from about 5% to about 15%, in view of the balance between conditioning benefits and product appearance such as being a single phase liquid without solid precipitate and/or having a clear or transparent appearance.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.1% to about 30%, preferably up to about 20% by weight of the composition.

### Soluble Silicone Compound

The compositions may further comprise a silicone compound which is soluble in the solvent system, for providing additional conditioning benefits while having desired product appearance such as being a single phase liquid without solid precipitate and/or having a clear or transparent appearance. Such soluble silicone compounds can be used at levels by weight of the composition of preferably from about 0.1% to about 30%, more preferably from about 0.5% to about 10%, still more preferably, from about 1% to about 10% for rinse-off use and from about 0.5% to about 5% for leave-on use.

Preferably, the soluble silicone compounds are those having an HLB value of about 8 or more, more preferably from about 9 to about 17, in view of the product appearance and the conditioning benefits.

Preferably, the soluble silicone compounds are those having a viscosity of about 10,000 cst or less, more preferably about 5,000 cst or less, in view of the compatibility with the anhydrous low viscosity product and/or for the product appearance.

Such soluble silicone compounds include, for example, PEG modified or PEG/PPG modified silicones, such as those having an INCI name: PEG-12 dimethicone (which are sold under the names, for example, Silsoft 875, Silsoft 870), those having an INCI name: PEG/PPG 20/23 dimethicone (which are sold under the names, for example, Silsoft 430), those having an INCI name: PEG-12 Dimethicone, those having an INCI name: PEG/PPG 20/23 Dimethicone, those having an INCI name: PEG/PPG 23/6 Dimethicone, those having an INCI name: PEG-10 Dimethicone, those having an INCI name: PEG-12 Dimethicone, and those having an INCI name: PEG-17 Dimethicone.

Such soluble silicone compounds also include, for example, those having an INCI name polysilicone-29 (which are sold under the names, for example, Silsoft CLX-E).

Such soluble silicone compounds also include, for example, those having an INCI name Quaternium-80 which is a quaternary organo-silicone (which are sold under the names, for example, Abil Quat 3270, Abil Quat 3272 and Abil Quat 3474).

Among such soluble silicone compounds, PEG modified, or PEG/PPG modified silicones and those having an INCI name polysilicone-29 are preferred over those having an INCI name Quaternium-80, in view of overall performance including product appearance.

### Other Silicone Compound

The compositions of the present invention may, or may not, contain other silicone compounds than those described above under “SOLUBLE SILICONE COMPOUNDS”. It is believed that the silicone compound can provide smoothness and softness on dry hair. The silicone compounds herein can be used at levels by weight of the composition of preferably from about 0.1% to about 30%, more preferably from about 1% to about 20%, still more preferably from about 5% to about 20%.

The silicone compounds useful herein, as a single compound, as a blend or mixture of at least two silicone compounds, or as a blend or mixture of at least one silicone compound and at least one solvent, have a viscosity of preferably from about 50 to about 2,000,000 mPa·s at 25° C.

The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used. Preferred polyalkyl siloxanes include, for example, polydimethylsiloxane, polydiethylsiloxane, and polymethylphenylsiloxane. Polydimethylsiloxane, which is also known as dimethicone, is especially preferred. These silicone compounds are available, for example, from the General Electric Company in their Viscasil® and TSF 451 series, and from Dow Corning in their Dow Corning SH200 series.

The above polyalkylsiloxanes are available, for example, as a mixture with silicone compounds having a lower viscosity. Such mixtures have a viscosity of preferably from about 1,000 mPa·s to about 100,000 mPa·s, more preferably from about 5,000 mPa·s to about 50,000 mPa·s. Such mixtures preferably comprise: (i) a first silicone having a viscosity of from about 100,000 mPa·s to about 30,000,000 mPa·s at 25° C., preferably from about 100,000 mPa·s to about 20,000,000 mPa·s; and (ii) a second silicone having a viscosity of from about 5 mPa·s to about 10,000 mPa·s at 25° C., preferably from about 5 mPa·s to about 5,000 mPa·s. Such mixtures useful herein include, for example, a blend of dimethicone having a viscosity of 18,000,000 mPa·s and dimethicone having a viscosity of 200 mPa·s available from GE Toshiba, and a blend of dimethicone having a viscosity of 18,000,000 mPa·s and cyclopentasiloxane available from GE Toshiba.

The silicone compounds useful herein also include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. The silicone gums are available, for example, as a mixture with silicone compounds having a lower viscosity. Such mixtures useful herein include, for example, Gum/Cyclomethicone blend available from Shin-Etsu.

Silicone compounds useful herein also include amino substituted materials. Preferred aminosilicones include, for example, those which conform to the general formula (I):

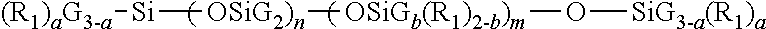

wherein G is hydrogen, phenyl, hydroxy, or C₁-C₅ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)2; —N(R₂)2; —N(R₂)3A; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion.

Highly preferred amino silicones are those corresponding to formula (I) wherein m=0, a=1, q=3, G=methyl, n is preferably from about 1500 to about 1700, more preferably about 1600; and L is —N(CH3)2 or —NH2, more preferably —NH2. Another highly preferred amino silicones are those corresponding to formula (I) wherein m=0, a=1, q=3, G=methyl, n is preferably from about 400 to about 600, more preferably about 500; and L is —N(CH3)2 or —NH₂, more preferably —NH₂. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone compounds include those having alkylamino substitutions as pendant groups of a silicone backbone. Highly preferred are those known as “amodimethicone”. Commercially available amodimethicones useful herein include, for example, BY16-872 available from Dow Corning. Some embodiments may include Silicone Quaternium-26.

The silicone compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben, imidazolidinyl urea, p-Hydroxyacetophenone; pH adjusting agents, such as citric acid, benzoic acid, sorbic acid, sodium citrate, succinic acid, phosphoric acid, sodium hydroxide, sodium carbonate; coloring agents, such as any of the FD&C or D&C dyes; perfumes; and sequestering agents, such as disodium ethylenediamine tetra-acetate; ultraviolet and infrared screening and absorbing agents such as benzophenones; and antidandruff agents such as zinc pyrithione, piroctone olamine. In some embodiments, the composition may be free of any silicone, sulfate, and/or paraben. The composition may further comprise a material selected from the group consisting of perfumes, preservatives, silicones, colorants, oils, acidifier, and combinations thereof.

### Low Melting Point Oil

The compositions may comprise one or more conditioning oils. Low melting point oils useful herein are those having a melting point of less than 25° C. The low melting point oil useful herein is selected from the group consisting of: hydrocarbon having from 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils including pentaerythritol tetraisostearate, trimethylol ester oils, citrate ester oils, and glyceryl ester oils; poly α-olefin oils such as polydecenes; and mixtures thereof. Additional oils may include polyester oil or mono-, di, tri-ether or ester including triglycerides, such as caprylic capric triglyceride or vegetable oils such as coconut oil, soybean oil, rapeseed oil, cocoa butter, olive oil, palm oil, rice bran oil, and mixtures thereof.

In some embodiments, a conditioning oil may have a hydrophilic-lipophilic balance (HLB) of less than about 10. In some embodiments, the oil may be a mono, di, or tri ester or ether where the monomer units have a carbon chain of C2 to C16, preferably C4 to C10, or more preferably C6 to C8. In some embodiments, the oil may be a polyester with the hydrophobic monomer units (linear or branched) having carbon chains shorter than C16, preferably shorter than C12. Commercially available oil examples include, but are not limited to, Myritol 318 from BASF (caprylic/capric triglyceride), Plantasil Micro from BASF (dicaprylyl ether in emulsion form (Dicaprylyl Ether (and) Decyl Glucoside (and) Glyceryl Oleate)); or Citropol 1A from P2 science (Polycitronellol Acetate).

### Examples

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

TABLE 1

| | S1 | S2 | S3 | S4 | S5 | S6 | S7 | S8 | S9 | S10 | S11 |
|---|---|---|---|---|---|---|---|---|---|---|---|
| Stearamidopropyl- | 5 | 1.33 | 3 | 5 | 5 | 5 | 3 | 3 | 4 | 4 | 4 |
| dimethylamine | | | | | | | | | | | |
| (SAPDMA) | | | | | | | | | | | |
| Myristyl Alcohol | 5.2 | 3.4 | 8.7 | 7.5 | — | 7.5 | — | 3.63 | 8.3 | 8.3 | 8.3 |
| (C14) | | | | | | | | | | | |
| Cetyl Alcohol | 6.5 | 4.3 | — | — | 7.5 | 7.5 | 5.27 | 5.27 | 1.5 | 1.5 | 1.5 |
| (C16) | | | | | | | | | | | |
| Stearyl Alcohol | — | — | — | — | — | — | 3.63 | — | — | — | — |
| (C18) | | | | | | | | | | | |
| Salicylic Acid | 1.5 | 1.5 | 0.9 | 1.5 | 1.5 | 1.5 | 0.9 | 0.9 | 1.2 | 1.2 | 1.2 |
| Dipropylene glycol | 78 | 42 | 87.4 | 84 | 86 | 76.5 | — | 87.2 | 73 | 63 | 75 |
| Pentylene glycol | — | — | — | — | — | — | 87.2 | — | — | — | — |
| Propylene Glycol | — | 28 | — | — | — | — | — | — | — | — | — |
| Abil Quat 3272 | — | — | — | — | — | — | — | — | — | 20 | — |
| (Mixture of 50% | | | | | | | | | | | |
| Quaternium-80 and | | | | | | | | | | | |
| 50% Propylene | | | | | | | | | | | |
| glycol) | | | | | | | | | | | |
| Silsoft 430 | — | — | — | — | — | — | — | — | 10 | — | — |
| Silsoft CLX-E | — | — | — | — | — | — | — | — | — | — | 10 |
| Caprylic capric | — | — | — | — | — | 2 | — | — | — | — | — |
| triglyceride | | | | | | | | | | | |
| Perfume/ | QS | QS | QS | QS | QS | QS | QS | QS | QS | QS | QS |
| Preservative | | | | | | | | | | | |
| Single phase no | Yes | Yes | Yes | Yes | Yes | Yes | Yes | Yes | Yes | Yes | Yes |
| precipitation at | | | | | | | | | | | |
| 15° C. | | | | | | | | | | | |
| % T | At | At | At | At | At | At | At | At | At | At | At |
| | least | least | least | least | least | least | least | least | least | least | least |
| | 80% | 80% | 80% | 80% | 80% | 80% | 80% | 80% | 80% | 80% | 80% |
| Viscosity | 1 to | 1 to | 1 to | 1 to | 1 to | 1 to | 1 to | 1 to | 1 to | 1 to | 1 to |
| | 100 | 100 | 100 | 100 | 100 | 100 | 100 | 100 | 100 | 100 | 100 |
| | cPs | cPs | cPs | cPs | cPs | cPs | cPs | cPs | cPs | cPs | cPs |
| Wet detangling | Good | Good | Good | Good | Good | Good | Good | Good | Good | Good | Good |
| performance | | | | | | | | | | | |

TABLE 2

| | C1 | C2 | C3 | C4 | C5 | C6 |
|---|---|---|---|---|---|---|
| Stearamidopropyldimethylamine | 5 | 5 | 10 | — | 3 | 1 |
| (SAPDMA) | | | | | | |
| Myristyl Alcohol (C14) | — | — | — | 10 | — | — |
| Cetyl Alcohol (C16) | — | — | — | — | — | — |
| Stearyl Alcohol (C18) | — | — | — | — | — | 19 |
| Salicylic Acid | — | — | — | — | 0.9 | — |
| Dipropylene glycol | 95 | — | 90 | 90 | 95.1 | 80 |
| Pentylene glycol | — | 95 | — | — | — | — |
| Perfume | — | — | — | — | 1 | — |
| Single phase no | yes | Yes | Yes | Yes | Yes | No |
| precipitation at 15° C. | | | | | | |
| % T | At least | At least | At least | At least | At least | Not |
| | 80% | 80% | 80% | 80% | 80% | applicable, |
| | | | | | | as not a |
| | | | | | | single |
| | | | | | | phase |
| Viscosity | 1 to | 1 to | 1 to | 1 to | 1 to | Not |
| | 100cPs | 100cPs | 100cPs | 100cPs | 100cPs | applicable, |
| | | | | | | as not a |
| | | | | | | single |
| | | | | | | phase |
| Wet detangling performance | Poor | Poor | Poor | Poor | Poor | Not |
| | | | | | | applicable, |
| | | | | | | as not a |
| | | | | | | single |
| | | | | | | phase |

TABLE 3

| | F-a | F-b |
|---|---|---|
| SAPDMA | | |
| BAPDMA | 1.3 | |
| Cetrimonium Chloride | | |
| Behentrimonium Chloride | | 1.3 |
| Behentrimonium methosulfate | | |
| Myristyl Alcohol (C14) | 8.7 | 8.7 |
| Cetyl Alcohol (C16) | | |
| Stearyl Alcohol (C18) | | |
| Salicylic Acid | 0.9 | 0.5 |
| Dipropylene glycol | 88 | 86 |
| Caprylic capric triglyceride | | |
| Silsoft 430 | | |
| Perfume/Preservative | QS | QS |
| Single phase no precipitation at | Y | Y |
| 15° C. | | |
| % T | At least 80% | At least 80% |
| Viscosity | 1 to 100 cPs | 1 to 100 cPs |
| Wet detangling performance | Good | Good |

The above hair care compositions in Tables 1-3 can be prepared by any conventional method well known in the art.

The above hair care compositions in Tables 1-3 are used by a method comprising the following steps:
Dispense the composition from a container;
Apply the composition to wet hair;
wherein, before applying to the wet hair, the method does not contain a step to mix the composition with water in a ratio more than 1:0.8, preferably more than 1:0.5.

The above hair care compositions in Tables 1 and 3 are examples of the present disclosure, and the hair care compositions in Table 2 are comparative examples.

The examples of the present disclosure provides a hair care composition: which provide, in addition to frizz reduction, good conditioning benefit especially during application such as the application to wet hair such as wet detangling, while not weighing down the hair and/or making the hair heavy and/or greasy; which contains reduced amount of C1-C3 mono-alcohol in view of reduced irritation and/or reduced dry perception; which is a single phase liquid without solid precipitate, and/or has a clear or transparent appearance; and which can be used in a convenient manner, for example, without such dilution with water before use.

For example, compared to the comparative examples C1-C5, the examples of the present disclosure provide improved conditioning benefit especially during application such as the application to wet hair such as wet detangling. Examples presented in C1-C5 do not contain either cationic surfactant or fatty alcohol to form a gel network structure in such a way to provide wet detangling performance. Compared to the comparative examples C6, the examples of the present disclosure provide single phase liquid without solid precipitate, and/or has a clear or transparent appearance.

### Test Methods

### Single Phase/No Precipitation Assessment

Sample is placed in a temperature controlled room at 15° C. The appearance is observed after the sample have equilibrated for more than 48 hours at that condition.

### Clarity Assessment—Measurement of % Transmittance (% T)

Composition clarity can be measured by % Transmittance. For this composition to be clear, the composition may be made without ingredients that would give the composition a hazy appearance such as silicones, opacifiers, non-silicone oils, micas, and gums. It is believed that adding these ingredients would not impact the nature of the compositions function, however these ingredients may obscure measurement of clarity by % Transmittance. A clear composition will generally be a single phase, and should remain clear even when shaken, while two-phase compositions will not be clear when shaken.

Clarity can be measured by % Transmittance (% T) using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to ensure no air bubbles are within the sample before the % T is measured by the spectrophotometer at 600 nm. An example of equipment used for measurement is X-Rite Ci7800 Bench Top Spectrophotometer. The compositions of the present invention may have a percent transmittance (% T) of at least about 80% transmittance at 600 nm.

### Rheology and Viscosity Method

Rheology and viscosity are used to evaluate and characterize product samples. The key rheology methods identified are mentioned below:
Shear Stress at 950s⁻¹ via flow curve: This is the method to ramp up shear rate logarithmically from 0.1 to 1000 s⁻¹ in 1 min using a cone & plate geometry, and to read the shear stress value a (Pa) at shear rate 950 s⁻¹.
Viscosity, measured is centipoise, cPs, by a Cone Plate Viscometer, such as Brookfield Viscometer RV-DV2TRV available from Brookfield Engineering Laboratories (USA) or other substitutable model known in the art. A cone such as CPA-41Z rotated at 1 rpm with sample volume 2 ml with a 3-minute measurement duration with the measurement read at the end of this 3 minutes. The exact selection of measurement conditions may be selected as needed by one skilled in the art.

For the inventive compositions, the viscosity may be below 100 cPs. For inventive compositions, when combined with water in a ratio of composition to water from about 1:1 to about 1:20, the shear stress may be at least 100 Pa at a shear rate of 950s⁻¹.

### Combinations

Embodiment 1. A hair care composition comprising:
(a) from about 1.2% to about 5% of a cationic surfactant;
(b) from about 3% to about 20% of a fatty alcohol selected from the group of consisting of myristyl alcohol, cetyl alcohol, and mixtures thereof,
(c) from about 50% to about 90% of a solvent system selected from the group consisting of di-propylene glycol, propylene glycol, pentylene glycol, hexylene glycol, 1,2-hexanediol, sugar alcohol, PEG-4, and mixtures thereof,
wherein the composition contains less than about 5% of a C1-C3 mono-alcohol;
wherein the composition further comprises (d) from about 0.5% to about 20% of a frizz reduction agent
   Embodiment 2. The hair care composition of Embodiment 1, wherein the frizz reduction agent is citric acid, succinic acid and/or at least one of those having the structure: having the structure:

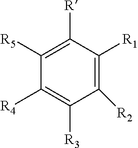

wherein R′ is —COOY, sulfonic acid, or —C═CH—COOY, Y is hydrogen or a metal ion, R₁, R₂, R₃, R₄, R₅ is hydrogen, methyl, ethyl, propyl, vinyl, allyl, methoxy, ethoxy, hydroxyl, halogen, sulfate, sulfonate, nitro, or —CH═CH—COOR, and wherein the frizz reduction agent is an acidic material and further wherein the frizz reduction agent has a % Protein binding higher than 20 and Molecular Volume lower than 500 and Partition coefficient octanol to water (log P) lower than 3 and hydrogen binding higher than 10 and pKa lower than 5.0, preferably wherein the frizz reduction agent is selected from the group consisting of citric acid, succinic acid, 2,4-Dihydroxybenzoic acid, 3-Hydroxybenzoic Acid, Gallic acid, 3-Aminobenzoic acid, 4-Aminobenzoic acid, 2,5-Dihydroxybenzoic acid, 3,4-Dihydroxybenzoic acid, 3,5-Dihydroxybenzoic acid, 2,6-Dihydroxybenzoic acid, 5-Chlorosalicylic acid, Salicylic acid, Trans-Ferulic Acid, p-Coumaric acid, 4-Hydroxybenzenesulphonic acid, 3-Chloro-4-hydroxybenzoic acid, 3,5-Dichloro-4-hydroxybenzoic acid, 2,5 Dihydroxyterephthalic acid, 3-Aminophenol, 3-Hydroxyanilinium chloride, 2-Aminophenol, 4-Aminophenol, N-4-Hydroxyphenylglycine, and mixtures thereof, preferably the frizz reduction agent is selected from the group consisting of salicylic acid, 5-chlorosalicylic acid, p-Coumeric acid, and ferulic acid, and mixtures thereof, more preferably the frizz reduction agent is salicylic acid.
Embodiment 3. The hair care composition of any of the preceding Embodiments, wherein the composition comprises from about 80% to about 90% of the solvent system.
Embodiment 4. The hair care composition of any of the preceding Embodiments, wherein the solvent system is selected from the group consisting of di-propylene glycol, propylene glycol, pentylene glycol, hexylene glycol, 1,2-hexanediol, and mixtures thereof.
Embodiment 5. The hair care composition of any of the preceding Embodiments, wherein the solvent system contains from about 50% to about 100%, preferably from about 75% to about 100%, by weight of the solvent system, of dipropylene glycol.
Embodiment 6. The hair care composition of any of the preceding Embodiments, wherein the total amount of the cationic surfactant and the fatty alcohol is from about 4.2% to about 22%, preferably from about 5% to about 15%.
Embodiment 7. The hair care composition of any of the preceding Embodiments, wherein the composition contains less than 1%, preferably less than 0.5%, more preferably less than 0.1%, still more preferably 0% of fatty alcohols having 18 or more carbons.
Embodiment 8. The hair care composition of any of the preceding Embodiments, wherein the fatty alcohol contains from about 25% to about 100%, preferably from about 30% to about 80%, by weight of the fatty alcohol, of myristyl alcohol.
Embodiment 9. The hair care composition of any of the preceding Embodiments, wherein the composition is anhydrous liquid, preferably the anhydrous liquid having a viscosity of from about 1 to about 300 cPs.
Embodiment 10. The hair care composition of any of the preceding Embodiments, wherein the composition is a single phase, and wherein the composition is clear or transparent.
Embodiment 11. The composition of any of the preceding Embodiments, further comprising from about 0.1% to about 30% of a silicone compound which is soluble to the solvent system, wherein the soluble silicone having an HLB value of about 8 or more and having a viscosity of about 10,000 cSt or less.
Embodiment 12. A method of use the composition of any of the preceding Embodiments, comprising the following steps:
Dispense the composition from a container;
Apply the composition to wet hair;
wherein, before applying to the wet hair, the method does not contain a step to mix the composition with water in a ratio more than 1:0.8, preferably more than 1:0.5.
   Embodiment 13. Use of the composition of any of the preceding Embodiments for frizz reduction.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair care composition comprising:
(a) from about 1.2% to about 5% of a cationic surfactant;
(b) from about 3% to about 20% of a fatty alcohol selected from the group of consisting of myristyl alcohol, cetyl alcohol, and mixtures thereof,
(c) from about 50% to about 90% of a solvent system selected from the group consisting of di-propylene glycol, propylene glycol, pentylene glycol, hexylene glycol, 1,2-hexanediol, sugar alcohol, PEG-4, and mixtures thereof,
wherein the composition contains less than about 5% of a C1-C3 mono-alcohol;
wherein the composition further comprises (d) from about 0.5% to about 20% of a frizz reduction agent.

2. The hair care composition of claim 1, wherein the frizz reduction agent is citric acid, succinic acid and/or at least one of those having the structure: 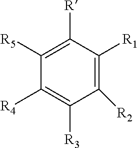
wherein R′ is —COOY, sulfonic acid, or —C═CH—COOY, Y is hydrogen or a metal ion, R₁, R₂, R₃, R₄, R₅ is hydrogen, methyl, ethyl, propyl, vinyl, allyl, methoxy, ethoxy, hydroxyl, halogen, sulfate, sulfonate, nitro, or —CH═CH—COOR, and wherein the frizz reduction agent is an acidic material and further wherein the frizz reduction agent has a % Protein binding higher than 20 and Molecular Volume lower than 500 and Partition coefficient octanol to water (log P) lower than 3 and hydrogen binding higher than 10 and pKa lower than 5.0.

3. The hair care composition of claim 2, wherein the frizz reduction agent is selected from the group consisting of citric acid, succinic acid, 2,4-Dihydroxybenzoic acid, 3-Hydroxybenzoic Acid, Gallic acid, 3-Aminobenzoic acid, 4-Aminobenzoic acid, 2,5-Dihydroxybenzoic acid, 3,4-Dihydroxybenzoic acid, 3,5-Dihydroxybenzoic acid, 2,6-Dihydroxybenzoic acid, 5-Chlorosalicylic acid, Salicylic acid, Trans-Ferulic Acid, p-Coumaric acid, 4-Hydroxybenzenesulphonic acid, 3-Chloro-4-hydroxybenzoic acid, 3,5-Dichloro-4-hydroxybenzoic acid, 2,5 Dihydroxyterephthalic acid, 3-Aminophenol, 3-Hydroxyanilinium chloride, 2-Aminophenol, 4-Aminophenol, N-4-Hydroxyphenylglycine, and mixtures thereof.

4. The hair care composition of claim 3, wherein the frizz reduction agent is selected from the group consisting of salicylic acid, 5-chlorosalicylic acid, p-Coumeric acid, and ferulic acid, and mixtures thereof.

5. The hair care composition of claim 4, wherein the frizz reduction agent is salicylic acid.

6. The hair care composition of claim 1, wherein the composition comprises from about 80% to about 90% of the solvent system.

7. The hair care composition of claim 1, wherein the solvent system is selected from the group consisting of di-propylene glycol, propylene glycol, pentylene glycol, hexylene glycol, 1,2-hexanediol, and mixtures thereof.

8. The hair care composition of claim 1, wherein the solvent system contains from about 50% to about 100%, by weight of the solvent system, of dipropylene glycol.

9. The hair care composition of claim 8, wherein the solvent system contains from about 75% to about 100%, by weight of the solvent system, of dipropylene glycol.

10. The hair care composition of claim 1, wherein the total amount of the cationic surfactant and the fatty alcohol is from about 5% to about 15%.

11. The hair care composition of claim 1, wherein the composition contains less than 1% of fatty alcohols having 18 or more carbons.

12. The hair care composition of claim 11, wherein the composition contains less than 0.5% of fatty alcohols having 18 or more carbons.

13. The hair care composition of claim 12, wherein the composition contains less than 0.1% of fatty alcohols having 18 or more carbons.

14. The hair care composition of claim 13, wherein the composition contains 0% of fatty alcohols having 18 or more carbons.

15. The hair care composition of claim 1, wherein the fatty alcohol contains from about 25% to about 100%, by weight of the fatty alcohol, of myristyl alcohol.

16. The hair care composition of claim 15, wherein the fatty alcohol contains from about 30% to about 80%, by weight of the fatty alcohol, of myristyl alcohol.

17. The hair care composition of claim 1, wherein the composition is anhydrous liquid.

18. The hair care composition of claim 1, wherein the composition is a single phase, and wherein the composition is clear or transparent.

19. The composition of claim 1, wherein the composition has a viscosity of from about 1 to about 300 cPs.

20. The composition of claim 1, further comprising from about 0.1% to about 30% of a silicone compound which is soluble to the solvent system, wherein the soluble silicone having an HLB value of about 8 or more and having a viscosity of about 10,000 cSt or less.

21. A method of use the composition of claim 1, comprising the following steps:
Dispense the composition from a container;
Apply the composition to wet hair;
wherein, before applying to the wet hair, the method does not contain a step to mix the composition with water in a ratio more than 1:0.8.

22. Use of the composition of claim 1 for frizz reduction.
